(19) United States
(12) Patent Application Publication
(10) Pub. No.: US 2015/0112443 A1
(43) Pub. Date: Apr. 23, 2015

(54) IMPLANTABLE BONE AUGMENT AND METHOD FOR MANUFACTURING AN IMPLANTABLE BONE AUGMENT

(71) Applicant: Frederik Gelaude, Leuven (BE)

(72) Inventor: Frederik Gelaude, Leuven (BE)

(73) Assignee: Mobelife N.V., Leuven (BE)

(21) Appl. No.: 14/399,152

(22) PCT Filed: May 14, 2012
(86) PCT No.: PCT/EP2012/058883

§ 371 Date: Dec. 19, 2014

(51) Int. Cl. A61B 19/00; G06F 17/50; A61F 2/30

(52) U.S. Cl. CPC A61B 19/50; A61F 2/30; G06F 17/50; A61F 2210/00
USPC 623/18.11; 700/98

(57) ABSTRACT

Method for manufacturing an implantable bone augment arranged to at least partially fit in a bone defect in a bone of a patient, wherein the method comprises the steps of: —providing a three-dimensional model of at least a part of the bone of the patient comprising the bone defect; —designing the shape and size of an augment based on the three-dimensional model, wherein a bone contacting surface of the augment is formed complementary to a corresponding outer surface of the bone, in particular a surface of the bone defect; —designing the body of the augment having the designed shape and size and having a porous microstructure, wherein the step of designing further comprises providing reinforcements in said body based on predicted loading conditions of the implanted augment, and; —manufacturing the designed augment.

The present invention relates to a method for manufacturing an implantable bone augment arranged to at least partially fit in a bone defect in a bone of a patient. The invention further relates to an implantable bone augment.

In bone and joint reconstructive surgery, often bone defects need to be filled. One option is the use of alloplastic (e.g. metal) bone augments. These augments typically have a standardized shape and are available in a series with different sizes. These augment shapes range from block to zeppelin-like. Based on preoperative Xray, or simply by intraoperative measurement, the surgeon selects the most adequate shape and size of augment. The bone is shaped using a reamer to accommodate the selected augment in the host bone.

In case of large or shape-complex defect cavities, multiple standard augments need to be used. Mostly a cement layer is put between the augments to try to generate a stable overall augmentation. The augments are typically produced as foam, resulting in uniform macro-level characteristics.

It is a drawback of these known augments that it may be necessary to ream, thus remove, health bone tissue to allow a close fit of the augment.

It is therefore a goal, among other goals, of the present invention to provide an improved, efficient and/or reliable method for manufacturing an implantable bone augment.

This goal, among other goals, is met by a method for manufacturing an implantable bone augment according to appended claims 1.

More in particular, this goal, among other goals, is met by method for manufacturing an implantable bone augment arranged to at least partially fit in a bone defect in a bone of a patient, wherein the method comprises the steps of:
providing a three-dimensional model of at least a part of the bone of the patient comprising the bone defect;
designing the shape and size of an augment based on the three-dimensional model, wherein a bone contacting surface of the augment is formed complementary to a corresponding outer surface of the bone, in particular a surface of the bone defect;
designing the body of the augment having the designed shape and size and having a porous microstructure, wherein the step of designing further comprises providing reinforcements in said body based on predicted loading conditions of the implanted augment, and;
manufacturing the designed augment.

According to the invention, the design of the augment is based on the geometry, i.e. the three-dimensional model, of the bone wherein the augment is to be implanted. An augment can therefore be made patient specific, such that reaming of the bone during the implant procedure can be minimized or even prevented at all. As the design of the augment, in particular the shape (outer contours) and the size, of the augment are based on the geometry of the bone, a close fit of the augment to the bone after implantation can be guaranteed.

The augment is thereto provided with, or designed to have, at least one bone contacting surface. The geometry of this outer surface of the augment is formed complementary to the geometry of a corresponding surface of the bone upon which the bone contacting surface is designed to engage, i.e. the geometries of the two surfaces arranged to contact in implanted situation are substantially equal. The geometry of the corresponding surface of the bone follows from the provided three-dimensional model of the bone. Preferably substantially a whole surface, or more preferably the whole surface, of the augment directed towards the bone in implanted situation is designed as a bone contacting surface to allow a stable rest of the augment on the bone.

The shape of the augment is inter alia based on the size of the defect, or malformation, the augment is designed to repair. The augment is therefore preferably shaped to closely fit in, preferably at least partially fill, more preferably fill, the defect. Even more preferably the augment is shaped to complement the bone to its original outer contours. This allows the impaired bone to be used in its intended manner again. The step of designing the shape and size of the augment may therefore comprise providing an outer surface substantially complementary to the original outer surface of the bone, or to at least closely match the surrounding bone to provide a smooth geometrical transition between the surrounding bone and the augment.

The shape can however also, or only, be dependent of additional implants that need to be implanted or other components, including other bones, adjacent to the augment in implanted situation. For instance, in case a cup needs to be placed in the acetabulum in a total hip arthroplasty, the boundaries or rim/dome of the acetabulum may be deteriorated. To allow a stable placement, i.e. good support, of this cup, a defect in the rim needs to be repaired. An augment according to the invention can then be designed to at the one hand fit in the defect and at the other hand receive and support an additional implantable component such as a cup. According to a preferred embodiment, the step of designing the shape and size of an augment furthermore comprises the step of providing a component contacting surface which is arranged to contact an additional implantable component. The component contacting surface is formed complementary to a corresponding surface of this component.

It should however be noted that in the invention is not limited to for instance augments in the hip for restoring the dome of the acetabulum. The augment, or implant as such, may for instance already include such a cup, base plate or other liner receiving feature. The component contacting surface is in that case cup shaped and for instance be provided with an acetabular liner. The augment or implant according to the invention can furthermore be used in other repair procedures, for instance in the shoulder joint. The component contacting surface can then arranged to form a backing, whereon a glenoid liner can be provided.

It is further important that the three-dimensional model of the bone resembles the bone in the state wherein the augment is to be implanted. It may for instance be possible that prior to implanting the augment, portions of the bone are reamed. The three-dimensional model is then preferably adapted accordingly, to allow the close fit of the augment to the reamed surface according to the invention.

After the size and shape of the augment, i.e. the outer contours of the augment are designed, a body having the designed size and shape is designed. The body of the augment according to the invention has a porous microstructure to facilitate bone ingrowth in the implanted situation.

The microstructure of the body preferably comprises a repeating microstructure. The microstructure preferable has interconnected struts for forming voids there between. It should be noted that the term struts as used herein is not limited to connecting structures having a particular cross-section. The struts of the microstructure may for instance have a circular, rectangular or even varying cross-section along their length.

To increase the applicability of the augment according to the invention, in particular with respect to augments having smaller sizes, the method further comprises the step of designing reinforcements based on predicted loading conditions, preferably loading conditions in the body of the augment. For instance in case that increased loads are expected due to the geometry of the body, i.e. the size and shape thereof, the body is reinforced to cope with these loading conditions. Locally reinforcing the body for instance allows the design of a body having thin portions, for instance border portions of the augment providing a smooth geometrical transition to surrounding bone structure. According to this aspect, the reinforcements locally reinforce thin portions of the augment. Predicting the loading conditions can for instance encompass determining the local thickness of the augment, or even the local amount of body material in the augment, and providing reinforcing structure based thereon, wherein the amount of reinforcing structure is inversely proportional to the local thickness, or amount of local material.

The reinforcements may further provide global support to the augment, for instance in terms of overall stiffness of the augment. This is of particular relevance for augments having a length which is large with respect to their height and width. Providing a reinforcing structure, for instance extending along at least a part of the entire length of the body, provides rigidity to the augment.

It should further be noted that the term loading conditions may relate to (local) stresses, strains or combinations thereof (for instance strain energy density). In the step of designing the reinforcements in the body, also load transfers through the body to for instance bone structures or additional components can be accounted for. It is of particular importance that reinforcements are provided to prevent local loading conditions, for instance stresses and strains, above a material failure loading value such that the augment will fail in implanted situation.

According to a preferred embodiment, the step of providing the three-dimensional model comprises the step of obtaining an image of the bone and defect therein. Digital patient-specific image information can be provided by any suitable means known in the art, such as for example a computer tomography (CT) scanner, a magnetic resonance imaging (MRI) scanner, an ultrasound scanner, or a combination of Roentgenograms. A summary of medical imaging has been described in “Fundamentals of Medical imaging”, by P. Suetens, Cambridge University Press, 2002.

For example, the step of obtaining an image of the bone and the defect therein may for example comprise the steps of obtaining 2D datasets of the bone and reconstructing a 3D virtual bone model from said 2D datasets. Indeed, the first step in a planning is the construction of a 3D virtual model of the bone. This reconstruction starts with sending a patient to a radiologist for scanning, e.g. for a scan that generates medical volumetric data, such as a CT, MRI scan or the like. The output of the scan can be a stack of two-dimensional (2D) slices forming a 3D data set. The output of the scan can be digitally imported into a computer program and may be converted using algorithms known in the field of image processing technology to produce a 3D computer model of a relevant bone. Preferably, a virtual 3D model is constructed from the dataset using a computer program such as Mimics™ as supplied by Materialise N.V., Leuven, Belgium. Computer algorithm parameters are based on accuracy studies, as for instance described by Gelaude at al. (2008; Accuracy assessment of CT-based outer surface femur meshes Comput. Aided Surg. 13(4): 188-199). A more detailed description for making a perfected model is disclosed in U.S. Pat. No. 5,768,134 entitled ‘Method for making a perfected medical model on the basis of digital image information of a part of the body’. Once the three-dimensional model of the bone is reconstructed for instance as disclosed in Gelaude et al. (2007; Computer-aided planning of reconstructive surgery of the innominate bone: automated correction proposals Comput. Aided Surg. 12(5): 286-94), the size and the shape of the augment can be designed based thereon as described above.

According to a further preferred embodiment, the step of manufacturing comprises using a three-dimensional printing technique, also referred to as rapid manufacturing technique, layered manufacturing technique, additive manufacturing technique or material deposition manufacturing technique.

Rapid manufacturing includes all techniques whereby an object is built layer by layer or point per point by adding or hardening material (also called free-form manufacturing). The best known techniques of this type are stereolithography and related techniques, whereby for example a basin with liquid synthetic material is selectively cured layer by layer by means of a computer-controlled electromagnetic beam; selective laser sintering, whereby powder particles are sintered by means of an electromagnetic beam or are welded together according to a specific pattern; fused deposition modelling, whereby a synthetic material is fused and is stacked according to a line pattern; laminated object manufacturing, whereby layers of adhesive-coated paper, plastic, or metal laminates are successively glued together and cut to shape with a knife or laser cutter; or electron beam melting, whereby metal powder is melted layer per layer with an electron beam in a high vacuum.

In particular embodiments, Rapid Prototyping and Manufacturing (RP&M) techniques, are used for manufacturing the augment of the invention. Rapid Prototyping and Manufacturing (RP&M) can be defined as a group of techniques used to quickly fabricate a physical model of an object typically using three-dimensional (3-D) computer aided design (CAD) data of the object. Currently, a multitude of Rapid Prototyping techniques is available, including stereo lithography (SLA), Selective Laser Sintering (SLS), Fused Deposition Modeling (FDM), foil-based techniques, etc. A common feature of these techniques is that objects are typically built layer by layer.

Stereo lithography (SLA), presently the most common RP&M technique, utilizes a vat of liquid photopolymer “resin” to build an object a layer at a time. On each layer, an electromagnetic ray, e.g. one or several laser beams which are computer-controlled, traces a specific pattern on the surface of the liquid resin that is defined by the two-dimensional cross-sections of the object to be formed. Exposure to the electromagnetic ray cures, or, solidifies the pattern traced on the resin and adheres it to the layer below. After a coat had been polymerized, the platform descends by a single layer thickness and a subsequent layer pattern is traced, adhering to the previous layer. A complete 3-D object is formed by this process.

Selective laser sintering (SLS) uses a high power laser or another focused heat source to sinter or weld small particles of plastic, metal, or ceramic powders into a mass representing the 3-dimensional object to be formed.

Fused deposition modeling (FDM) and related techniques make use of a temporary transition from a solid material to a liquid state, usually due to heating. The material is driven through an extrusion nozzle in a controlled way and deposited in the required place as described among others in U.S. Pat. No. 5,141,680.

Foil-based techniques fix coats to one another by means of gluing or photo polymerization or other techniques and cut the object from these coats or polymerize the object. Such a technique is described in U.S. Pat. No. 5,192,539.

Typically RP&M techniques start from a digital representation of the 3-D object to be formed, in this case the design of the augment. Generally, the digital representation is sliced into a series of cross-sectional layers which can be overlaid to form the object as a whole. The RP&M apparatus uses this data for building the object on a layer-by-layer basis. The cross-sectional data representing the layer data of the 3-D object may be generated using a computer system and computer aided design and manufacturing (CAD/CAM) software.

The implantable augment of the invention may be manufactured in different materials. Typically, only materials that are biocompatible (e.g. USP class VI compatible) with the human body are taken into account. Preferably the augment is formed from a heat-tolerable material allowing it to tolerate high-temperature sterilization. In the case SLS is used as a RP&M technique, the surgical template may be fabricated from a polyamide such as PA 2200 as supplied by EOS, Munich, Germany or any other material known by those skilled in the art may also be used.

According to a further preferred embodiment the step of providing reinforcements in the body comprises adapting local material properties of the body, such as material type and/or Young's modulus. By locally providing a different material type, changes in local loading conditions, in particular local concentrations, can be coped with. With the term different material types, also the same materials having different mechanical properties, such as the Young's modulus are meant. Although it is possible to locally manufacture the body of, or design the body to locally have, a different material type having different mechanical properties, it is preferred to modify the mechanical properties of the material throughout the body in accordance with predicted loading conditions. It is for instance possible to modify the Young's modulus of a material using laser technology.

According to a further preferred embodiment the step of providing reinforcements in the body comprises locally adapting the design of the microstructure, in particular the local density of the microstructure of the body. Based on the predicted loading conditions, the thickness of for instance the struts in the microstructure is adapted locally. In particular, in case high local loads are predicted, for instance based on simulations as will be discussed in more detail below, the local thickness of the struts of the microstructure can be increased to cope with these increased loads. It is however also possible to locally adapt the repeating microstructure in terms of size and shape, i.e. the design. For instance, the local number of interconnected struts may be increased in case increased local loads are predicted. Also, by decreasing the local scale of the repeating microstructure, while keeping the diameters of the struts substantial equal, the local density is increased accordingly. It goes without saying that in case decreased local loads are predicted or calculated by simulation, a decrease in density is also possible to save on material volume.

According to this preferred embodiment at least a part of the reinforcements comprises a non-solid porous microstructure. This improves bone ingrowth characteristics for cell size and the structural stiffness of the part (locally and globally). To further reduce for instance the weight, it is also possible that the reinforcements are formed by a non-solid porous microstructure only. In this particular embodiment, the body is formed to be completely porous, wherein the reinforcements are formed by increasing the local density of the microstructure or by locally adapting the mechanical properties of the microstructure.

According to a further preferred embodiment at least a part of the reinforcements comprises a solid structure. This increases the reinforcing properties, i.e. the capability to withstand increased loading conditions for instance due to the geometrical design of the body. Preferably the solid structure is manufactured from, or designed to be manufactured from, the same material as the porous microstructure. This is in particular advantageous in case the augment is manufactured using a three-dimensional printing technique as described above. In this embodiment, the solid structure can be interpreted as a porous microstructure having a density of 100%.

According to a further preferred embodiment of the method according to the invention the porous microstructure comprises struts forming voids there between, wherein the microstructure adjacent to the solid structure comprises struts with a widening diameter, i.e. increased cross-sectional areas, towards the solid structure. This improves the load transfer between porous structure and the solid structure, such that excess concentrations of stresses and strains are prevented.

It is further possible to design other special features, next to for instance the contacting surfaces, in the body. It is for instance possible to include a cavity in the body to improve bone ingrowth. These special features, including cavities, which have a weakening effect on the body as such, are then reinforced by the reinforcing structure.

A further preferred embodiment of the method according to the invention further comprises the step of designing connecting means for connecting the augment to the bone, for instance in the form of spikes and/or holes for receiving screws, wherein the reinforcements are provided in the body based on predicted loading conditions of the connecting means. In order to connect the augment to the bone, or perhaps other surrounding structures such as additional implants, connecting means or connectors may be necessary. According to this aspect, the reinforcements are arranged to reinforce special features of the augment such as connecting means, for instance in the form of screw holes. As these connecting means influence the loading conditions in implanted conditions, the reinforcements are arranged to strengthen the body of the augment where needed. In particular around screw holes high local stresses and strains occur. Providing reinforcements around the screw holes prevents failure of the body due to these increased loading conditions.

Preferably the step of designing the connecting means comprises identifying bone structures in the three-dimensional model suitable for receiving connecting means, for instance based on bone quality analysis, and designing the connecting means to cooperate with said identified bone structures. Bone quality analysis can for instance be a CT-based/Houndsfield-based analysis to assess the cortical bone thickness or the trabecular bone Young's modulus. A complementary quantitative tool previously developed by the applicant for assessing the degree of bone loss in the acetabulum is also preferred (Gelaude et al, Quantitative Computerized Assessment of the Degree of Acetabular Bone Deficiency: Total radial Acetabular Bone Loss (TrABL), Adv Orthop. 2011; 2011:494382). Based on the three-dimensional model, trajectories of for instance screws can be determined which extend through high quality bone to allow a firm connection between the augment and the bone. The connecting means, in this example the screw holes, can then be designed in the body to allow the screws to be inserted in the bone in accordance with the determined trajectories, thereby fixating the augment to the bone. Also based on these trajectories and the designed connecting means, the loading conditions as a result of connecting the augment to the bone can be predicted or even calculated by simulation.

A further preferred embodiment of the method according to the invention further comprises the step of simulating the augment in implanted condition using the design of the augment and the three-dimensional model of the bone for determining said predicted loading conditions. As a digital three-dimensional, preferably meshed, model of the bone is already available for the design of the augment and the design of the model is also in digital form using CAD, a combinational model of the augment and the bone can be made. For instance by using the Finite Element Method (FEM) by applying boundary conditions mimicking the daily external load on the bone, this combinational model can be used to calculate the local loading conditions, for instance the local stress and strains. The reinforcements in the body are then provided based on these simulated loading conditions.

It should however be noted that the invention is not limited to simulation based on FEM only. It is for instance possible to predict, calculate and/or deduct the loading conditions using a musculoskeletal model (MSM) based on simulation of body and joint kinematics (Range-of-Motion of the joint) and dynamics (muscle and joint forces), the effect of joint centre displacement on forces and the effect of joint centre displacement and therewith muscle lengthening (tensioning) on forces.

The method preferably further comprises the step of repeating the steps of designing the body and simulating the loading conditions in an iterative process for reducing the simulated local loads in the body at least below a material failure loading value. In case in an earlier step according to the invention loads are predicted being higher than the maximum tolerable load of the used material, the body is locally reinforced. The augment with the provided reinforcement is evaluated again to check whether all loading conditions are within an acceptable range, i.e. below a material failure loading value. In case still higher loads are calculated, the reinforcements are increased in size or the material properties are adapted accordingly. Eventually, the body of the augment, in particular the reinforcements thereof, will be adapted to the loading conditions in (simulated) implanted situation.

The method more preferably further comprises the step of determining the simulated loading conditions in the surrounding bone and repeating the steps of designing the body and simulating the loading conditions in the surrounding in an iterative process for obtaining an optimal loading conditions, for instance substantially even loading conditions, in the surrounding bone. Not only the loading conditions in the augment can be determined and adapted by providing reinforcements, also the load distribution in the surrounding bone can be determined using simulation, in particular FEM. It is therefore possible to adapt the augment, in particular the placement of the reinforcements thereof, to provide an optimal, for instance a substantially even, loading distribution in the bone adjacent the augment. Both high loads and low loads are to be prevented according to Wolffs law (Wolff, Das Gesetz der Transformation der Knochen—1892. Reprint: Pro Business, Berlin 2010, ISBN 978-3-86805-648-8). This prevents resorption of the bone due to stress shielding and also bone loss due to peaks in local loads. Preferably, the loading distribution in the surrounding bone is engineered by designing the augment to be within a normal loading range of bone.

It should be noted that also the loading conditions, for instance the loading distribution, in other adjacent components can be taken into account. It is for instance possible to include the loading distribution in additional implants or connecting means in the form of for instance screws in the simulation. The augment can then be engineered to prevent local peaks in loading in such components.

According to a further preferred embodiment of the method according to the invention, the step of designing the shape and size of the augment comprises forming a first bone contacting surface complementary to a corresponding outer surface of the bone defect and forming a second bone contacting surface complementary to a corresponding outer surface of an intact section of the bone of the patient. The augment is hereby designed to rests on, or to be supported by, also intact bone structures. The first bone contacting surface ensures a proper fit in the defect to allow an efficient fill thereof, while the second contacting surface ensures an even more stable fit of the augment in implanted situation.

The invention further relates to an implantable bone augment comprising a body having a porous microstructure and having a size and shape arranged to at least partially fit in a bone defect in a bone of a patient, wherein the body comprises a bone contacting surface formed complementary to a corresponding outer surface of the bone of the patient, in particular a surface of the bone defect, wherein at least a part of the body has different material types for forming a reinforcing structure of the body. As described above, an augment according to the invention can be designed to have thin sections, while failure of these sections is prevented. The same advantages and features as described in relation with the method above, apply mutatis mutandis to the augment as such, and vice versa. Preferably, at least a part of the body comprises a porous microstructure having a different design for forming the reinforcing structure. As described above, it is for instance possible to adapt the density of the microstructure and/or the architecture of the struts.

According to a further preferred embodiment of the augment according to the invention, at least a part of the reinforcing structure extends through an internal region of the body. The reinforcements, or reinforcing structure, hereby at least partially extend through, or are located in, the body of the augment. The porous microstructure of the body hereby at least partially encloses the reinforcements. This improves the support provided by the reinforcements.

It should be noted that the invention is not limited to a single reinforcing structure in the body. It is for instance possible to provide a plurality of reinforcing structures in the body if the loading conditions require so.

A further preferred embodiment of the implantable bone augment according to the invention further comprises connecting means for connecting the augment to the bone of patient, for instance in the form of spikes and/or holes for receiving screws, wherein the reinforcing structure is arranged for reinforcing the body at least adjacent said connecting means. This ensures that loads resulting from the connection between the augment and the bone do not lead to local failure of the porous microstructure. Preferably the connecting means comprise a plurality of screw receiving holes extending through the body, wherein the reinforcing structure at least extends between the screw receiving holes. This distributes the loads throughout the body, while at the same time providing support to the body.

A further preferred embodiment of the implantable bone augment according to the invention comprises a first bone contacting surface formed complementary to a corresponding outer surface of the bone defect and a second bone contacting surface formed complementary to a corresponding outer surface of an intact section of the bone of the patient. The first bone contacting surface ensures a proper fit in the defect to allow an efficient fill thereof, while the second contacting surface ensures an even more stable fit of the augment in implanted situation.

The present invention is further illustrated by the following Figures, which show a preferred embodiment of the device according to the invention, and are not intended to limit the scope of the invention in any way, wherein:

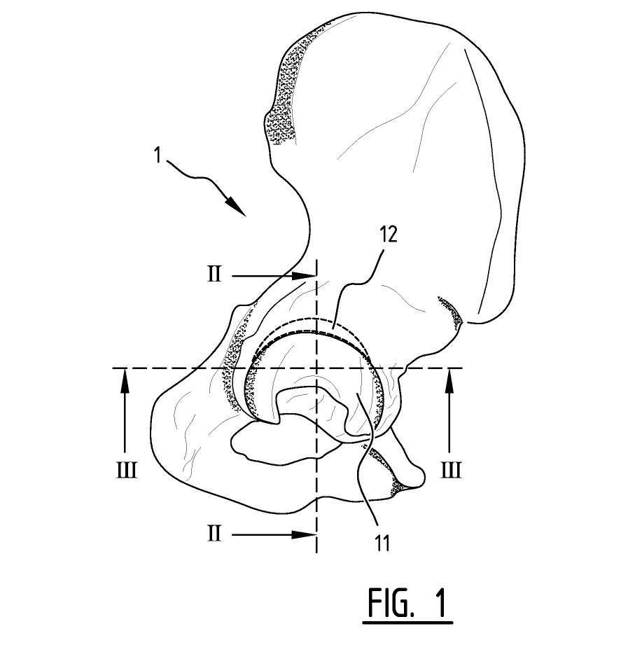

FIG. 1 schematically shows the hemi-pelvis in a perspective view on the acetabulum;

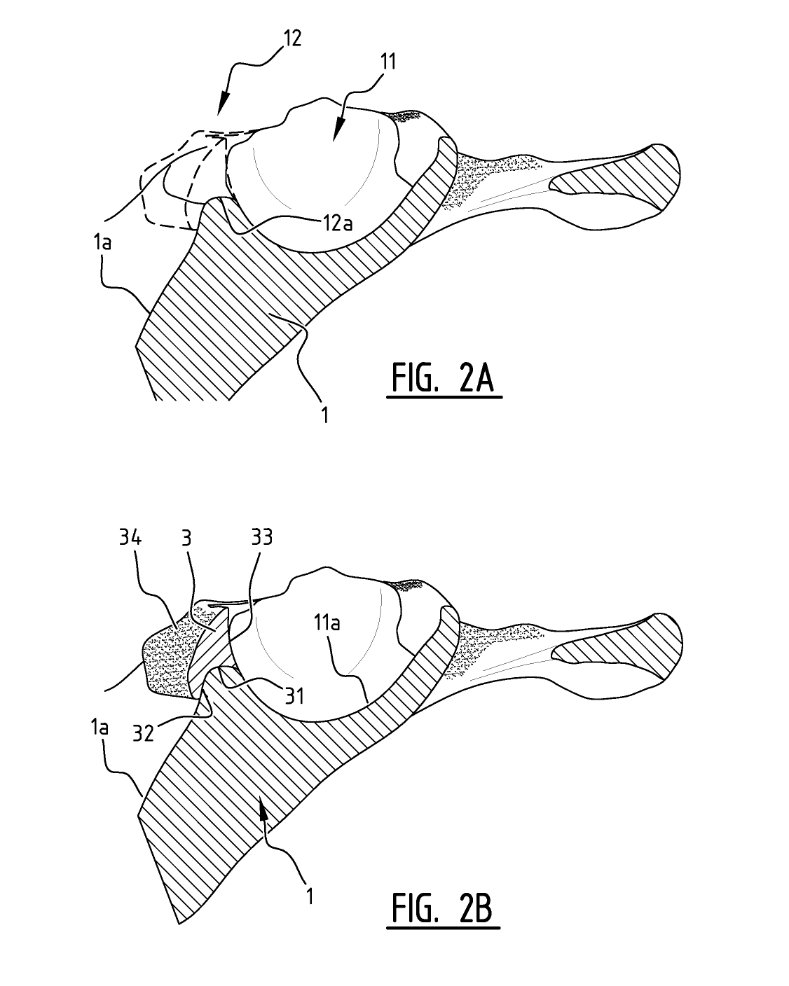

FIGS. 2a and 2b are cut-away views along line II in FIG. 1 showing the step of designing the shape and size of implant;

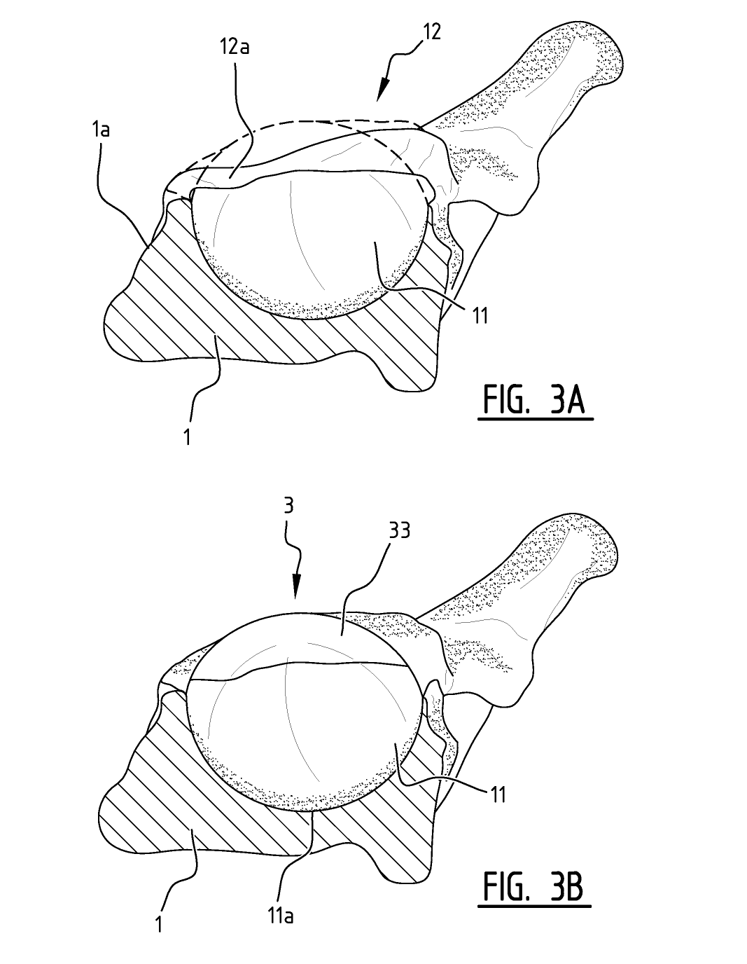

FIGS. 3a and 3b are cut-away views along line III in FIG. 1 corresponding to FIGS. 2a and 2b;

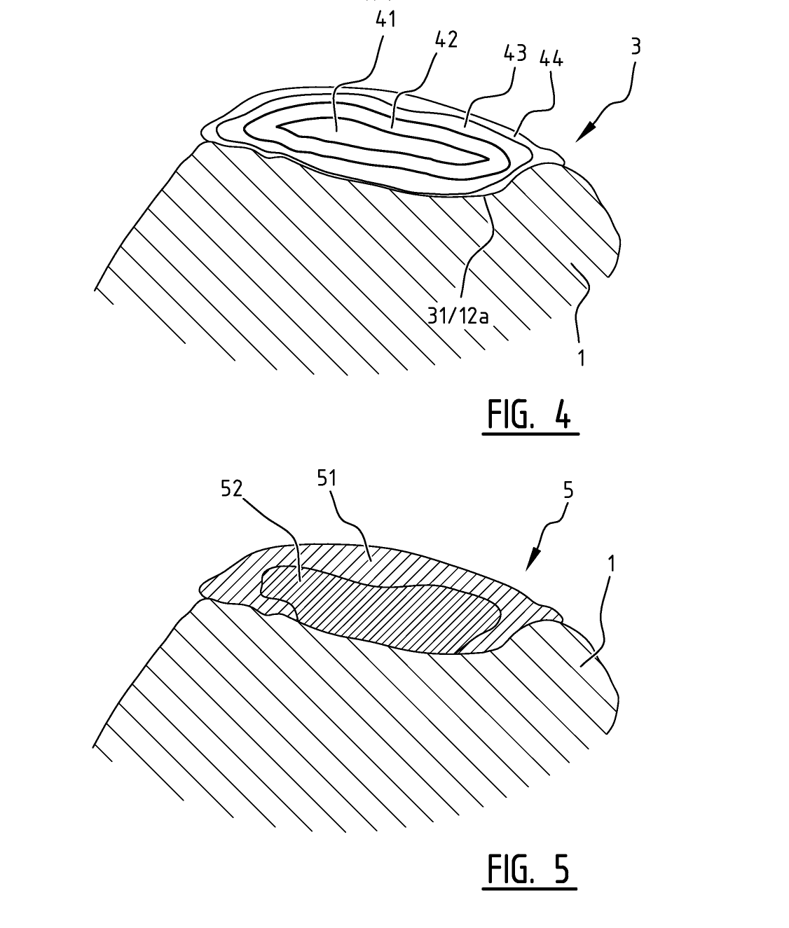

FIG. 4 schematically shows a simulated load distribution in a cross-section of the augment;

FIG. 5 schematically shows the design of a reinforcing structure based on the load distribution of FIG. 4;

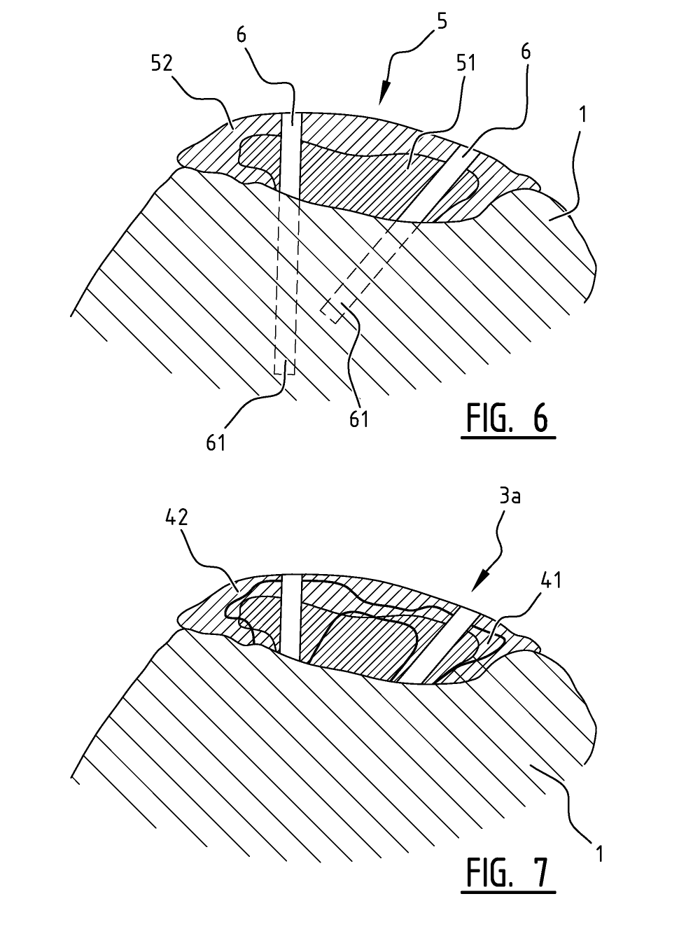

FIG. 6 schematically shows in cross-section the design of screw holes in the augment;

FIG. 7 schematically shows a simulated load distribution in a cross-section of the augment as designed in FIG. 6;

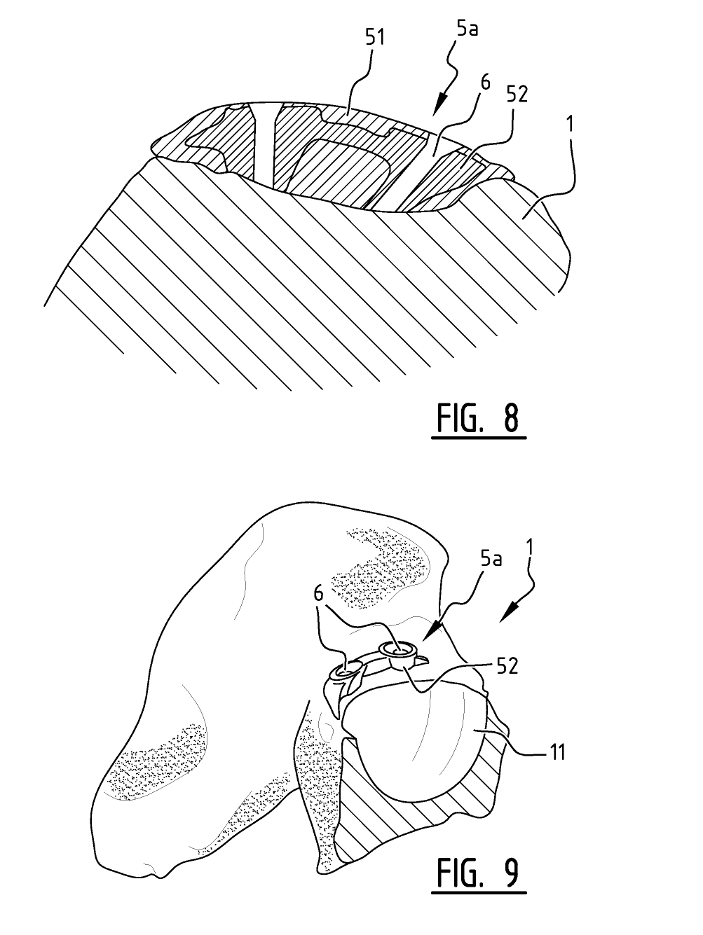

FIGS. 8 and 9 schematically show the design of a reinforcing structure based on the load distribution of figure in cross-section, respectively in perspective view, and;

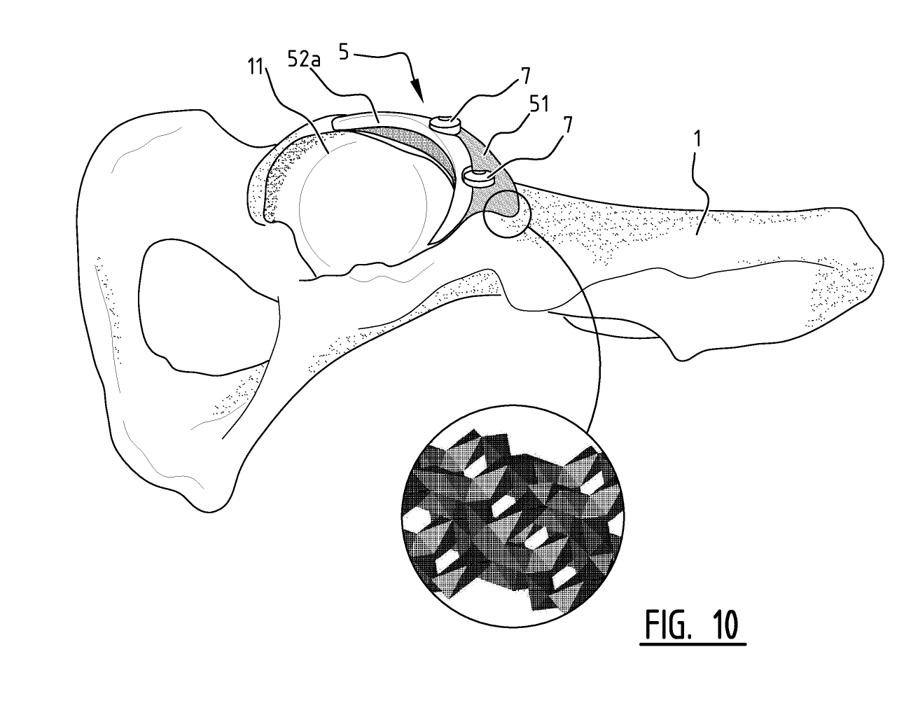

FIG. 10 schematically shows in perspective a second embodiment of the augment in connected state on the bone, and in a cut-away view.

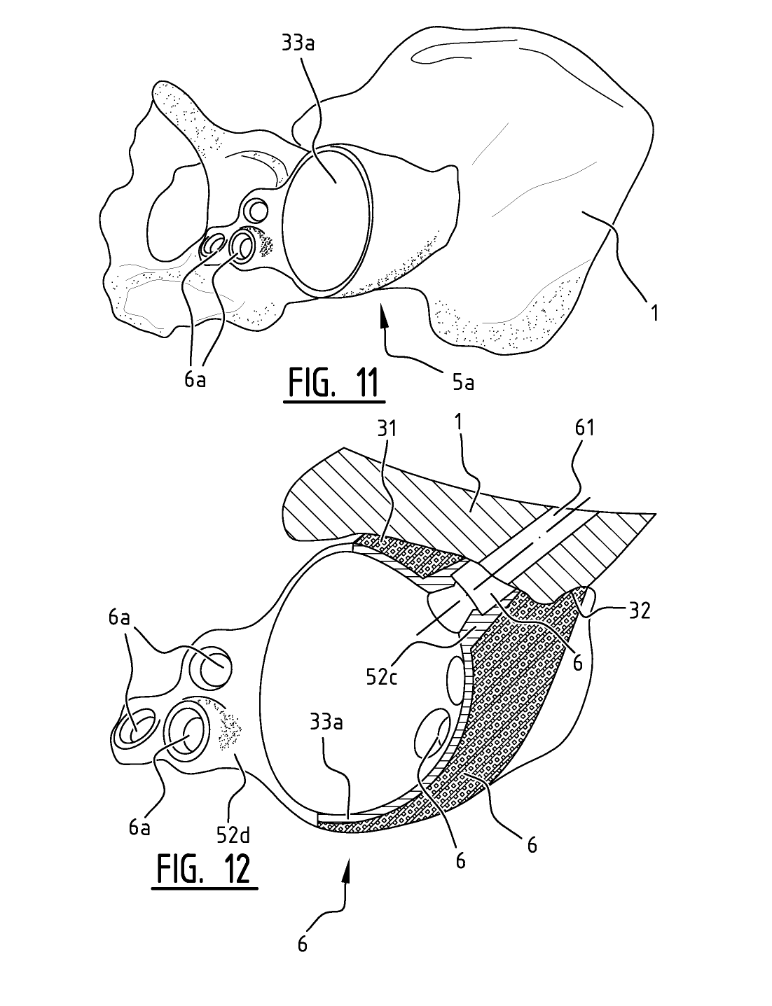

FIGS. 11 and 12 schematically show a third embodiment of the implant in perspective view, respectively in cross-section.

In this example, the process of manufacturing an augment for a patient having defect in the acetabular rim in the ilium region is elucidated. In order to design the augment, a digital three-dimensional meshed model is obtained from the defect and the surrounding bone. In this example, the model is constructed from a series of two-dimensional parallel scan planes obtained by a CT-scan. The slices are combined in order to obtain a three-dimensional model, as is disclosed in more detail in Gelaude at al. (2008; Accuracy assessment of CT-based outer surface femur meshes Comput. Aided Surg. 13(4): 188-199). In this example, a model of the whole pelvis 1 is obtained as shown in FIG. 1. The defect 12 at the rim of the acetabulum is indicated with dashed lines in FIG. 1.

Based on the model of the bone 1, which provides detailed geometrical surface data of the outer surface 1a of the bone and in particular of the outer surface 12a in the region of the defect 12, the shape and size of an augment can be designed, as shown in dashed lines in FIGS. 2a and 3a.

The body 3 of the augment, see FIGS. 2b and 3b, is designed to a have a bone contacting surface 31 which is formed complementary to a surface 12a of the defect 12. In other words, the surface 31 is designed such that surfaces 12a and 31 extend adjacently in close contact, although some play may be required to allow placement of the augment in the bone. This can for instance be achieved by designing the bone contacting surface 31 to have the same geometry as the surface 12a, for instance by using the coordinates of the geometry of the surface 12a in the three-dimensional meshed model. The surface 31 is designed to contact the whole outer surface of the defect 12, such that the body 3 of the augment can fill the defect.

The body 3 is further designed to have a second bone contacting surface 32 which is designed to contact an outer surface 1a of the intact bone 1. This surface 32 is formed in the same way as the first bone contacting surface 31 and provides extra stability.

The augment is designed to restore the integrity of the acetabulum. With specific reference to FIGS. 2b and 3b, it is therefore important that the surface 11a is restored, for instance for receiving the head of the femur or a cup arranged for receiving the head of a femur prosthesis. An outer restoring surface 33 of the body 3 is therefore designed to allow a smooth transition between the surface 11a of the acetabulum 11 and the augment 3. More in particular, the restoring surface 33 is designed to restore the surface 11a of the acetabulum 11 without the defect 12.

The other surface 34 of the body 3 is designed to connect the bone contacting surfaces 31, 32 and restoring surface 31 while ensuring that the body 3 has enough structural rigidity. Moreover, also the surface 34 is designed to have a smooth transition between the surface 34 and the outer surface 1a of the bone 1.

The design of the shape and size of the body 3 of the augment is therefore based on the geometry of the bone 1, the defect 12 therein and the restoring function of the augment, in this example restoring surface 11a of the acetabulum 11.

According to the invention, the augment is designed to have a body 3 having a porous microstructure. This improves the bone ingrowth in implanted situation. In order to allow the use of such a porous microstructure in an augment designed to fill a relative small defect as in the current example, a reinforcement structure is included in the body 3.

Thereto, in a next step, a three-dimensional combined meshed model of the bone and the design of the body 3 is created which can be used in a Finite Element Method-analysis. In this model, the body 3 of the augment is designed to have a standard repeating microstructure (see for instance the insert in FIG. 10) and patient specific boundary conditions (i.e. external loading) are applied to the model. As a result thereof, a distribution of the stresses in the body 3 in implanted situation can be calculated as shown in FIG. 4. Region 41 corresponds to a region in the body having the highest stresses, while the regions 42-44 experience successive lower stresses. Based on this calculation, it is determined which loading conditions, i.e. stresses in this example, fall in an allowable range for the microstructure. In other words, it is determined if specific regions in the body 3 experience loads being higher than a failure load of the microstructure. These regions are designed as reinforcing structure 52, see FIG. 5. The remaining regions 51 of the design 5 are designed to be formed by the microstructure. In this example, the reinforcing structure 51 is manufactured from the same material as the microstructure 51, wherein the reinforcing structure 51 is designed to be solid.

In a next step, it is determined how the augment 5 is to be connected to the bone 1, for instance using a FEM-analysis. It is for instance possible that the augment can be fixed to the bone in a stable manner without the use of any connecting means. In that case, the design of the augment is complete. In this example however, screws are used, such that the trajectories 61 in the bone 1 need to be determined. The trajectories 61 are based on a bone quality analysis, such that the trajectories 61 extend through bone regions of good quality, preferably without intersecting. Based on these most optimal trajectories 61, screw holes 6 are designed in the augment 5. In this example, the screw holes 6 extend through the reinforcing structure 51, although this is not necessary.

Based on this adapted design of the augment 5, a new combinational model 3a is assembled and again the loading distribution throughout the body is calculated. In the region indicated with 41 the highest stresses occur, while in the region 42 lower stresses are calculated. In this calculation, also the influence of the screws (not shown) on the distribution of the loads is included. In this simulation, it is also possible to include the influence of other components, for instance the cup to be implanted in the acetabulum. Moreover, it is possible to calculate the loading distribution throughout the bone 1 surrounding the augment 5 to determine whether loads (or the absence thereof) may lead to bone degradation. If this is the case, the reinforcing structure 52 can be adapted accordingly to locally increase or decrease the load in the bone.

Based on the outcome of this step, the reinforcing structure 52 is adapted to a new design of the augment 5a, see FIG. 8. In FIG. 9, the reinforcing structure 52 and the screw holes 6 of the augment 5a are shown on the bone 1 (the porous structure 51 is not shown in this figure).

It should be noted that although in this example two calculations of the load distributions are made, i.e. after the design of the contours of the body and after the design of the screw holes, it is also possible to first design the body of the augment, including any special features such as the screw holes, and subsequently calculate the load distribution.

It is further possible to design the body with the reinforcing structure(s) in an iterative process. For instance, also the design as shown in FIG. 8 can be subjected to another calculation of the loading distribution to check whether peak loads above the failure load are to be expected in implanted situation. The design can then be adapted accordingly and subjected to another calculation. The same iterative process can be used to engineer an optimal loading distribution in the bone and/or additional components as described above.

Until now, the design and simulation of the design can be digitally, for instance using Computer Aided Design. When the design of the augment is finished, the design is manufactured using an additive manufacturing technique.

In FIG. 10, an augment 5 according to the invention is shown in implanted situation. The augment 5 comprises a body having a porous structure 51 wherein a part of the reinforcing structure extends (not visible, as the structure 52 extends internally in the porous structure 51). This internal reinforcing structure 51 corresponds the structure 52 as shown in FIG. 9. An external part 52a of the reinforcing structure is visible in FIG. 10. The reinforcing structure formed as flange 52a is arranged to cope with loads resulting from the placement of a cup for receiving a head of a head of a femoral prosthesis. In FIG. 10, the screws 7 extending through the screw holes 6 and through the trajectories 61 are furthermore visible.

In FIGS. 11 and 12 a variant of the implant according to the invention is shown. In this example, the implant 5a is designed to receive, for instance via a liner, a head of a femoral prosthesis itself, without implantation of an additional cup. The surface 33a of the body is therefore cup shaped. A bone contacting surface 31 is again formed to mate with the outer surface of the bone defect, while an additional surface 32 is again arranged to mate with a surface of a healthy section of the bone 1. A flange 52d is furthermore provided to accommodate screw holes 6a. After the design of the contours of the augment, the reinforcement structures are designed.

In this case, it is clear that the flange-like structure 52d of the body and the surface 33a need reinforcement in view of their function. In other words, no simulations are needed to determine that these sections are to be designed as a solid in view of the predicted loading conditions. It may however be advantageous to for instance determine the thickness of these sections using a simulation, for instance FEM. The same applies to the conically shaped reinforcements 52c enclosing the screw holes 6 (which are again designed on the basis of optimal trajectories 61 as discussed above). The reinforcements 52c are formed to enclose the screw holes, wherein the reinforcing structure has a tapering shape towards the bone 1. The rest of the implant 5 is formed as a porous structure 51.

The present invention is not limited to the embodiment shown, but extends also to other embodiments falling within the scope of the appended claims. It is for instance clear that also in the embodiment of FIGS. 11 and 12, simulations can be used to further specify the design of the reinforcing structure in the augment. Moreover, the applicability of the implant is not limited to the hip joint only and can be used in other bones which need to be repaired. It is also possible to add additional components to the augment after and/or during manufacturing, such as for instance liners.

1. Method for manufacturing an implantable bone augment arranged to at least partially fit in a bone defect in a bone of a patient, wherein the method comprises the steps of:
providing a three-dimensional model of at least a part of the bone of the patient comprising the bone defect;
designing the shape and size of an augment based on the three-dimensional model, wherein a bone contacting surface of the augment is formed complementary to a corresponding outer surface of the bone, in particular a surface of the bone defect;
designing the body of the augment having the designed shape and size and having a porous microstructure, wherein the step of designing further comprises providing reinforcements in said body based on predicted loading conditions of the implanted augment, and;
manufacturing the designed augment.

2. Method according to claim 1, wherein the step of manufacturing comprises using a three-dimensional printing technique.

3. Method according to claim 1 or 2, wherein the step of providing reinforcements in the body comprises adapting local material properties of the body, such as material type and/or Young's modulus.

4. Method according to any of the preceding claims, wherein the step of providing reinforcements in the body comprises adapting the local density of the microstructure of the body.

5. Method according to any of the preceding claims, wherein at least a part of the reinforcements comprises a non-solid porous microstructure.

6. Method according to any of the preceding claims, wherein at least a part of the reinforcements comprises a solid structure.

7. Method according to claim 6, wherein the porous microstructure comprises struts forming voids there between, wherein the microstructure adjacent to the solid structure comprises struts with a widening diameter towards the solid structure.

8. Method according to any of the preceding claims, further comprising the step of designing connecting means for connecting the augment to the bone, for instance in the form of spikes and/or holes for receiving screws, wherein the reinforcements are provided in the body based on predicted loading conditions of the connecting means.

9. Method according to claim 8, wherein the step of designing the connecting means comprises identifying bone structures in the three-dimensional model suitable for receiving connecting means, for instance based on bone quality analysis, and designing the connecting means to cooperate with said identified bone structures.

10. Method according to any of the preceding claims, further comprising the step of simulating the augment in implanted condition using the design of the augment and the three-dimensional model of the bone, for instance using the Finite Element Method, for determining said predicted loading conditions, wherein the reinforcements in the body are provided based on the simulated loading conditions.

11. Method according to claim 10, further comprising the step of repeating the steps of designing the body and simulating the loading conditions in an iterative process for reducing the simulated local loads in the body at least below a material failure loading value.

12. Method according to claim 10 or 11, further comprising the step of determining the simulated loading conditions in the surrounding bone and repeating the steps of designing the body and simulating the loading conditions in the surrounding in an iterative process for obtaining optimal loading conditions in the surrounding bone.

13. Method according to any of the preceding claims, wherein the step of designing the shape and size of the augment comprises forming a first bone contacting surface complementary to a corresponding outer surface of the bone defect and forming a second bone contacting surface complementary to a corresponding outer surface of an intact section of the bone of the patient.

14. Implantable bone augment comprising a body having a porous microstructure and having a size and shape arranged to at least partially fit in a bone defect in a bone of a patient, wherein the body comprises a bone contacting surface formed complementary to a corresponding outer surface of the bone of the patient, in particular a surface of the bone defect, wherein at least a part of the body has different material properties for forming a reinforcing structure of the body.

15. Implantable bone augment according to claim 14, wherein at least a part of the body comprises a porous microstructure having a different density for forming a reinforcing structure of the body.

16. Implantable bone augment according to claim 14 or 15, wherein at least a part of the reinforcing structure comprises a non-solid porous microstructure.

17. Implantable bone augment according to claim 14, 15 or 16, wherein at least a part of the reinforcing structure comprises a solid structure.

18. Implantable bone augment according to claim 17, wherein the porous microstructure comprises struts forming voids there between, wherein the microstructure adjacent to the solid structure comprises struts with a widening diameter towards the solid structure.

19. Implantable bone augment according to any of the preceding claims, wherein at least a part of the reinforcing structure extends through an internal region of the body.

20. Implantable bone augment according to any of the preceding claims, further comprising connecting means for connecting the augment to the bone of patient, for instance in the form of spikes and/or holes for receiving screws, wherein the reinforcing structure is arranged for reinforcing the body at least adjacent said connecting means.

21. Implantable bone augment according to claim 20, wherein the connecting means comprise a plurality of screw receiving holes extending through the body, wherein the reinforcing structure at least extends between the screw receiving holes.

22. Implantable bone augment according to any of the preceding claim comprising a first bone contacting surface formed complementary to a corresponding outer surface of the bone defect and a second bone contacting surface formed complementary to a corresponding outer surface of an intact section of the bone of the patient.
